(19) United States
(12) Patent Application Publication
(10) Pub. No.: US 2009/0198271 A1
(43) Pub. Date: Aug. 6, 2009

(54) ELECTRODE BASED FILTER

(75) Inventor: Yossi Gross, Moshav Mazor (IL)

Correspondence Address: DARBY & DARBY P.C., P.O. BOX 770, Church Street Station, New York, NY, 10008-0770, US

(73) Assignee: RAINBOW MEDICAL LTD., Herzliya (IL)

(21) Appl. No.: 12/361,949

(22) Filed: Jan. 29, 2009

Related U.S. Application Data
(60) Provisional application No. 61/025,133, filed on Jan. 31, 2008.

(51) Int. Cl. A61F 2/01

(52) U.S. Cl. 606/200

(57) ABSTRACT

Apparatus is described including a filter, configured to be implanted in a blood vessel of a subject. An electrode is configured to be disposed in a vicinity of the blood vessel. A control unit breaks up matter that aggregates on the filter, by driving the electrode to drive a current into the blood vessel and configuring the signal to increase nitric oxide (NO) secretion by the wall. Other embodiments are also described.

### CROSS-REFERENCES TO RELATED APPLICATIONS

The present application claims the benefit of U.S. Provisional Patent Application 61/025,133 to Gross, filed Jan. 31, 2008, entitled “Electrode based filter,” which is incorporated herein by reference.

### FIELD OF THE INVENTION

The present invention generally relates to implanted medical apparatus. Specifically, the present invention relates to apparatus and methods for cleaning implanted filters.

### BACKGROUND OF THE INVENTION

Filters are typically implanted into patients' blood vessels to prevent emboli from migrating through the bloodstream and occluding the supply of blood to vital organs.

US Patent Application Publication 2007/0196428 to Glauser et al. describes medical devices having a catalyst, which is described as being capable of catalyzing the generation of nitric oxide, attached to the medical device, and methods of treating a vascular condition using the devices.

US Patent Application Publication 2007/0248676 to Stamler et al. describes a method for introducing into a patient a device of which at least a portion includes a prophylactic or therapeutic amount of a nitric oxide adduct. The nitric oxide adduct can be present in a matrix coating on a surface of the medical device; coated per se on a surface of the medical device; directly or indirectly bound to reactive sites on a surface of the medical device; or at least a portion of the medical device can be formed of a material, such as a polymer, which includes the nitric oxide adduct. Also described is a method for preventing adverse effects associated with the use of a medical device in a patient by locally administering a nitric oxide adduct to the site of contact of said device with any internal tissue.

PCT Publication WO 00/002501 to Benjamin et al. describes intravascular stents that comprise copper, which leads to the generation of nitric oxide (in vivo) to prevent platelet activation.

PCT Publication WO 07/013065 to Gross describes apparatus, including a bifurcation stent comprising one or more electrodes, the stent configured to be placed in a primary passage and a secondary passage of a blood vessel, and a control unit, configured to drive the electrodes to apply a signal to a wall of the blood vessel, and to configure the signal to increase nitric oxide (NO) secretion by the wall.

U.S. Pat. No. 6,865,416 to Dev et al. describes methods for inducing or increasing the vasodilation of a vessel. The patent further describes methods for inducing or increasing the flow of fluid through a vessel. An electrical impulse is applied to the vessel in order to induce or increase vessel vasodilation or to induce or increase the flow of fluid through the vessel. In an embodiment described in the '416 patent, a double-balloon catheter system incorporating electroporation technology is used to apply the electrical impulse endoluminally.

The following references may be of interest:

European Patent Application Publication EP 0 109 935 A1 to Charmillot et al.

U.S. Pat. No. 5,324,323 to Bui

U.S. Pat. No. 5,645,839 to Chobanian et al.

U.S. Pat. No. 5,669,924 to Shaknovich

U.S. Pat. No. 5,800,502 to Boutos

U.S. Pat. No. 5,900,433 to Igo et al.

U.S. Pat. No. 5,904,712 to Axelgaard

U.S. Pat. No. 6,038,485 to Axelgaard

U.S. Pat. No. 6,058,331 to King

U.S. Pat. No. 6,086,527 to Talpade

U.S. Pat. No. 6,200,259 to March

U.S. Pat. No. 6,347,247 to Dev et al.

U.S. Pat. No. 6,463,323 to Conrad-Vlasak et al.

U.S. Pat. No. 6,810,286 to Donovan et al.

U.S. Pat. No. 6,824,561 and US Patent Application Publication 2004/0039417 to Soykan et al.

U.S. Pat. No. 6,845,267 to Harrison et al.

U.S. Pat. No. 6,871,092 to Piccone

U.S. Pat. No. 6,939,345 to KenKnight et al.

U.S. Pat. No. 7,082,336 to Ransbury

U.S. Pat. No. 7,090,648 to Sackner

U.S. Pat. No. 7,206,637 to Salo

U.S. Pat. No. 7,229,403 to Schock et al.

US Patent Application Publication 2002/0103454 to Sackner et al.

US Patent Application Publication 2003/0036773 to Whitehurst et al.

US Patent Application Publication 2003/0204206 to Padua et al.

US Patent Application Publication 2004/0106954 to Whitehurst et al.

US Patent Application Publication 2006-0229677 to Moffit

US Patent Application Publication 2006/0276844 to Alon et al.

PCT Publication WO 04/014456 to Allen et al.

PCT Publication WO 06/064503 to Belsky et al.

PCT Publication WO 06/094273 to White et al.

PCT Publication WO 06/123346 to Alon et al.

PCT Publication WO 07/064895 to Meyerhoff et al.

PCT Publication WO 07/106533 to Stern et al.

PCT Publication WO 07/113833 to Cahan et al.

PCT Publication WO 08/100390 to Walker

“Preparation and characterization of implantable sensors with nitric oxide release coatings,” by MC Frost et al., Microchemical Journal Vol: 74 Issue: 3, June, 2003 pp: 277-288

“Improving the Thromboresistivity of Chemical Sensors via Nitric Oxide Release: Fabrication and in Vivo Evaluation of NO-Releasing Oxygen-Sensing Catheters,” by MH Schoenfisch et al., Anal. Chem., 72 (6), 1119-1126, 2000

“Endogenous and Exogenous Nitric Oxide Protect Against Intracoronary Thrombosis and Reocclusion After Thrombolysis,” by Sheng-Kun Yao et al., Circulation. 1995;92:1005-1010

“Improving the biocompatibility of in vivo sensors via nitric oxide release,” by Jae Ho Shin et al., Analyst, 2006, 131, 609-615

### SUMMARY OF THE INVENTION

In some embodiments of the present invention, a filter is implanted in a blood vessel of a subject. An electrode is implanted in a vicinity of the blood vessel, and a control unit breaks up matter that aggregates on the filter, by driving the electrode to drive a current into the blood vessel and configuring the signal to increase nitric oxide (NO) secretion by the wall.

Typically, by driving the current, the control unit breaks up one or more emboli that aggregate on the filter, fibrotic material that adheres to the filter, and/or platelets that aggregate on the filter.

In some embodiments of the invention, a filter having two or more moving parts is implanted in a blood vessel of a subject. An electrode is implanted in a vicinity of the blood vessel, and a control unit moves the moving parts with respect to each other by driving the electrode to drive a current into the blood vessel that dilates the blood vessel by increasing nitric oxide (NO) secretion by the wall. Typically, the control unit thereby breaks up matter that aggregates on the filter by moving the moving parts with respect to each other due to the dilation of the blood vessel. Alternatively or additionally, the control unit moves the moving parts with respect to each other by driving the electrode to drive a current into the blood vessel that constricts the blood vessel. For example, the control unit may induce one or more cycles of dilation and contraction of the blood vessel, so as to jitter the filter and break up the aggregated matter.

For some applications, the moving parts are simply opposite ends of a structure associated with the filter (e.g., the filter itself), and the dilation or contraction of the blood vessel flexes the structure, thereby breaking up the matter that aggregated on the filter.

The filters described herein are suitable for short-term use (e.g., use during and shortly following a medical procedure), and are more typically used in chronic implantations, e.g., for periods from weeks to years.

There is therefore provided, in accordance with an embodiment of the invention, apparatus, including:

a filter, configured to be implanted in a blood vessel of a subject;

an electrode, configured to be disposed in a vicinity of the blood vessel; and

a control unit, configured to break up matter that aggregates on the filter, by driving the electrode to drive a current into the blood vessel and configuring the signal to increase nitric oxide (NO) secretion by the wall.

In an embodiment, the blood vessel includes an artery that supplies a brain of the subject, and the filter is configured to prevent emboli from occluding a flow of blood to the brain.

In an embodiment, the matter includes an embolus, and the control unit is configured to break up the embolus by driving the electrode to apply the current to the wall of the blood vessel.

In an embodiment, the matter includes fibrotic material that adheres to the filter, and the control unit is configured to break up the fibrotic material by driving the electrode to apply the current to the wall of the blood vessel.

In an embodiment, the matter includes platelets, and the control unit is configured to break up the platelets by driving the electrode to apply the current to the wall of the blood vessel.

In an embodiment, the electrode is configured to be disposed on the filter.

In an embodiment, the electrode is configured to be disposed inside the blood vessel.

In an embodiment, the electrode is configured to be disposed outside the blood vessel.

In an embodiment, the electrode is configured to be disposed in a wall of the blood vessel.

In an embodiment, the apparatus includes a detector configured to detect an aggregation of the matter on the filter, and the control unit is configured to drive the electrode to apply the current in response to the detector detecting the aggregation of the matter.

In an embodiment, the detector includes a deflection gauge, configured to sense a level of deflection of a portion of the apparatus due to aggregation of the matter on the filter.

In an embodiment, the detector is configured to detect the aggregation of the matter in response to a changed blood flow characteristic due to the aggregation of the matter on the filter.

In an embodiment, the control unit is configured to drive the electrode to apply the current in accordance with a duty cycle.

In an embodiment, the control unit is configured to set the duty cycle such that the current is applied during fewer than 10 minutes of at least one hour of a day.

In an embodiment, the control unit is configured to set the duty cycle such that the current is applied at least once during each of six hours of a day.

In an embodiment, the control unit is configured to break up the matter by driving the electrode to apply a current having an amplitude of between 1 mA and 5 mA.

In an embodiment, the control unit is configured to break up the matter by driving the electrode to apply a current having an amplitude of between 2 mA and 3 mA.

In an embodiment, the control unit is configured to break up the matter by driving the electrode to apply a current having a frequency of between 5 Hz and 20 Hz.

In an embodiment, the control unit is configured to break up the matter by driving the electrode to apply a current having a frequency of between 10 Hz and 15 Hz.

In an embodiment, the control unit is configured to break up the matter by driving the electrode to apply a current having two pulses to eight pulses per cardiac cycle.

In an embodiment, the control unit is configured to break up the matter by driving the electrode to apply a current having three pulses to five pulses per cardiac cycle.

There is further provided, in accordance with an embodiment of the invention, apparatus, including:

a filter having two or more moving parts and configured to be implanted in a blood vessel of the subject;

an electrode, configured to be disposed in a vicinity of the blood vessel; and

a control unit, configured to move the moving parts with respect to each other by driving the electrode to drive a current into the blood vessel that changes a diameter of the blood vessel.

In an embodiment, the blood vessel includes an artery that supplies a brain of the subject, and the filter is configured to prevent emboli from occluding a flow of blood to the brain.

In an embodiment, the electrode is configured to be disposed on the filter.

In an embodiment, the electrode is configured to be disposed inside the blood vessel.

In an embodiment, the electrode is configured to be disposed outside the blood vessel.

In an embodiment, the electrode is configured to be disposed in a wall of the blood vessel.

In an embodiment, the control unit is configured to drive the electrode to apply the current in accordance with a duty cycle.

In an embodiment, the apparatus includes a detector configured to detect an aggregation of the matter on the filter, and the control unit is configured to drive the electrode to apply the current in response to the detector detecting the aggregation of the matter.

In an embodiment, the control unit is configured to drive the electrode to drive a current into the blood vessel that dilates the blood vessel by increasing nitric oxide (NO) secretion by a wall of the blood vessel.

In an embodiment, the control unit is configured to dilate the blood vessel by driving the electrode to apply a current having an amplitude of between 1 mA and 5 mA.

In an embodiment, the control unit is configured to dilate the blood vessel by driving the electrode to apply a current having an amplitude of between 2 mA and 3 mA.

In an embodiment, the control unit is configured to dilate the blood vessel by driving the electrode to apply a current having a frequency of between 5 Hz and 20 Hz.

In an embodiment, the control unit is configured to dilate the blood vessel by driving the electrode to apply a current having a frequency of between 10 Hz and 15 Hz.

In an embodiment, the control unit is configured to dilate the blood vessel by driving the electrode to apply a current having two pulses to eight pulses per cardiac cycle of the subject.

In an embodiment, the control unit is configured to dilate the blood vessel by driving the electrode to apply a current having three pulses to five pulses per cardiac cycle of the subject.

In an embodiment, the control unit is configured to drive the electrode to drive a current into the blood vessel that constricts the blood vessel.

In an embodiment, the control unit is configured to constrict the blood vessel by driving the electrode to apply a current having a frequency of between 40 Hz and 70 Hz.

In an embodiment, the control unit is configured to constrict the blood vessel by driving the electrode to apply a current having an amplitude of between 5 mA and 20 mA.

In an embodiment, the control unit is configured to constrict the blood vessel by driving the electrode to apply a current having an amplitude of between 8 mA and 15 mA.

In an embodiment, the control unit is configured to constrict the blood vessel by driving the electrode to apply a current having ten pulses to twenty pulses per cardiac cycle of the subject

In an embodiment, the control unit is configured to constrict the blood vessel by driving the electrode to apply a current having thirteen pulses to seventeen pulses per cardiac cycle of the subject.

In an embodiment, the control unit is configured to break up matter that aggregates on the filter by moving the moving parts with respect to each other.

In an embodiment, the matter includes an embolus, and the control unit is configured to break up the embolus by moving the moving parts with respect to each other.

In an embodiment, the matter includes fibrotic material that adheres to the filter, and the control unit is configured to break up the fibrotic material by moving the moving parts with respect to each other.

In an embodiment, the matter includes platelets, and the control unit is configured to break up the platelets by moving the moving parts with respect to each other.

There is yet further provided, in accordance with an embodiment of the invention, a method, including:

implanting a filter in a blood vessel; and

breaking up matter that aggregates on the filter by driving a current into the blood vessel that increases nitric oxide (NO) secretion by the wall.

In an embodiment, the blood vessel includes an artery that supplies a brain of the subject, and implanting the filter includes preventing emboli from occluding a flow of blood to the brain by implanting the filter in the artery that supplies the brain.

In an embodiment, the matter includes an embolus that is filtered by the filter, and breaking up the matter includes breaking up the embolus.

In an embodiment, the matter includes fibrotic material that adheres to the filter, and breaking up the matter includes breaking up the embolus.

In an embodiment, the matter includes platelets that aggregate on the filter, and breaking up the matter includes breaking up the platelets.

In an embodiment, driving the current includes driving the current via an electrode that is coupled to the filter.

In an embodiment, driving the current includes implanting an electrode inside the blood vessel and driving the current via the electrode.

In an embodiment, driving the current includes implanting an electrode outside the blood vessel in a vicinity of the blood vessel and driving the current via the electrode.

In an embodiment, driving the current includes implanting an electrode in a wall of the blood vessel and driving the current via the electrode.

In an embodiment, driving the current includes driving the current in accordance with a duty cycle.

In an embodiment, the method includes detecting an aggregation of matter on the filter, driving the current includes driving the current in response to the detection of the aggregation of matter on the filter.

In an embodiment, driving the current includes driving a current having an amplitude of between 1 mA and 5 mA.

In an embodiment, driving the current includes driving a current having an amplitude of between 2 mA and 3 mA.

In an embodiment, driving the current includes driving a current having a frequency of between 5 Hz and 20 Hz.

In an embodiment, driving the current includes driving a current having a frequency of between 10 Hz and 15 Hz.

In an embodiment, driving the current includes driving a current having two pulses to eight pulses per cardiac cycle of the subject.

In an embodiment, driving the current includes driving a current having three pulses to five pulses per cardiac cycle of the subject.

There is also provided, in accordance with an embodiment of the invention, a method, including:

implanting, in a blood vessel of a subject, a filter having two or more moving parts; and

moving the moving parts with respect to each other by driving a current into the blood vessel that changes a diameter of the blood vessel.

In an embodiment, the blood vessel includes an artery that supplies a brain of the subject, and implanting the filter includes preventing emboli from occluding a flow of blood to the brain by implanting the filter in the artery that supplies the brain.

In an embodiment, driving the current includes driving the current via an electrode that is coupled to the filter.

In an embodiment, driving the current includes implanting an electrode inside the blood vessel and driving the current via the electrode.

In an embodiment, driving the current includes implanting an electrode outside the blood vessel in a vicinity of the blood vessel and driving the current via the electrode.

In an embodiment, driving the current includes implanting an electrode in a wall of the blood vessel and driving the current via the electrode.

In an embodiment, driving the current includes driving the current in accordance with a duty cycle.

In an embodiment, the method includes detecting an aggregation of matter on the filter, driving the current includes driving the current in response to the detection of the aggregation of matter on the filter, and moving the moving parts with respect to each other includes breaking up the matter.

In an embodiment, driving the current includes driving a current into the blood vessel that dilates the blood vessel by increasing nitric oxide (NO) secretion by a wall of the blood vessel.

In an embodiment, driving the current includes driving a current having an amplitude of between 1 mA and 5 mA.

In an embodiment, driving the current includes driving a current having an amplitude of between 2 mA and 3 mA.

In an embodiment, driving the current includes driving a current having a frequency of between 5 Hz and 20 Hz.

In an embodiment, driving the current includes driving a current having a frequency of between 10 Hz and 15 Hz.

In an embodiment, driving the current includes driving a current having two pulses to eight pulses per cardiac cycle of the subject.

In an embodiment, driving the current includes driving a current having three pulses to five pulses per cardiac cycle of the subject.

In an embodiment, driving the current includes driving a current into the blood vessel that constricts the blood vessel.

In an embodiment, driving the current includes driving a current having a frequency of between 40 Hz and 70 Hz.

In an embodiment, driving the current includes driving a current having an amplitude of between 5 mA and 20 mA.

In an embodiment, driving the current includes driving a current having an amplitude of between 8 mA and 15 mA.

In an embodiment, driving the current includes driving a current having ten pulses to twenty pulses per cardiac cycle of the subject.

In an embodiment, driving the current includes driving a current having thirteen pulses to seventeen pulses per cardiac cycle of the subject.

In an embodiment, moving the moving parts with respect to each other includes breaking up matter that aggregates on the filter.

In an embodiment, the matter includes an embolus that is filtered by the filter, and breaking up the matter includes breaking up the embolus.

In an embodiment, the matter includes fibrotic material that adheres to the filter, and breaking up the matter includes breaking up the fibrotic material.

In an embodiment, the matter includes platelets that aggregate on the filter, and breaking up the matter includes breaking up the platelets.

There is additionally provided, in accordance with an embodiment of the invention, apparatus, including:

a filter having two or more moving parts and configured to be implanted in a blood vessel of the subject; and

a control unit, configured to clean the filter while the filter is in the subject, by moving the moving parts with respect to each other.

The present invention will be more fully understood from the following detailed description of embodiments thereof, taken together with the drawings, in which:

### BRIEF DESCRIPTION OF THE DRAWINGS

FIG. 1 is a schematic illustration of a filter implanted in a blood vessel of a subject, in accordance with an embodiment of the present invention;

FIG. 2 is an exploded view of a filter having two or more moving parts, in accordance with an embodiment of the present invention; and

FIGS. 3A-B are schematic illustrations of the filter having two or more moving parts implanted in a blood vessel.

### DETAILED DESCRIPTION OF EMBODIMENTS

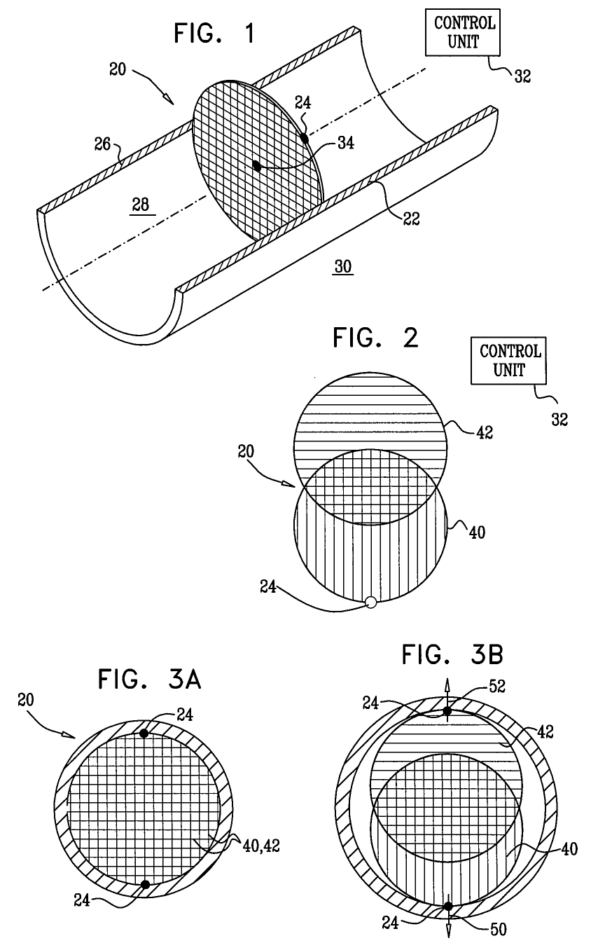

Reference is now made to FIG. 1, which is a schematic illustration of a filter 20 implanted in a blood vessel 22 of a subject. An electrode 24 is implanted in a vicinity of the blood vessel. A control unit 32 breaks up matter that aggregates on the filter by driving the electrode to drive a current into the blood vessel and configuring the signal to increase nitric oxide (NO) secretion by the wall. Typically, the nitric oxide breaks up the matter that has aggregated on the filter. In some embodiments, the matter is broken up in accordance with the techniques described in WO 07/013065 to Gross, which is incorporated herein by reference.

In some embodiments, blood vessel 22 is an artery, such as a caroti dartery that supplies the subject's brain. In such embodiments, filter 20 is typically implanted to prevent emboli from occluding blood flow to the brain. In an embodiment, blood vessel 22 is the aorta (for example, the ascending aorta), and filter 20 breaks up matter that aggregates on the filter and only allows the resultant smaller particles to pass through the filter and continue distally.

In some embodiments, filter 20 collects one or more emboli, and control unit 32 breaks up the emboli by driving the current into blood vessel 22. Alternatively or additionally, the control unit breaks up fibrotic material, and/or platelets that adhere to the filter.

In some embodiments (as shown), electrode 24 is coupled to filter 20. Alternatively or additionally, the electrode is implanted in a wall 26 of the blood vessel, within the lumen 28 of the blood vessel, and/or outside the blood vessel, in a vicinity 30 of the blood vessel.

In some embodiments, control unit 32 drives electrode 22 in accordance with a duty cycle. For example, the control unit may set the duty cycle such that the current is applied during fewer than 10 minutes of at least one hour of a day. Alternatively, the control unit sets the duty cycle such that the current is applied at least once during each of six hours of a day. Alternatively or additionally, a detector 34 detects an aggregation of matter on the filter, and control unit 32 drives the electrode to apply the current in response to the detector detecting the aggregation of the matter. For example, detector 34 may be a deflection gauge that detects the aggregation of matter on the filter by detecting deflection of a portion of the filter. Alternatively or additionally, the detector detects the aggregation of matter by detecting flow characteristics of blood flowing through the filter.

In some embodiments, control unit 32 breaks up the matter by driving electrode 24 to apply a current having an amplitude of between 1 mA and 5 mA, e.g. 2 to 3 mA. For some applications the current has a frequency of between 5 Hz and 20 Hz, e.g. 10 to 15 Hz. In some embodiments, the current has two pulses to eight pulses per cardiac cycle of the subject, e.g., three to five pulses.

Reference is now made to FIG. 2, which is an exploded view of filter 20 having two or more moving parts 40 and 42, in accordance with an embodiment of the present invention. Typically, control unit 32 is configured to move the moving parts with respect to each other by driving electrode 24 to drive a current into the blood vessel that dilates the blood vessel by increasing nitric oxide (NO) secretion by the wall. Further typically, the current for dilating the blood vessel has the same parameters as those of the current described above with respect to FIG. 1. Typically the control unit is configured to break up matter that aggregates on the filter by moving the moving parts with respect to each other. Alternatively or additionally, filter 20 is placed at the bifurcation of a second artery from a first artery (for example in a position to filter blood entering a carotid artery from the aorta), and movement of the moving parts disturbs any matter that has aggregated on the filter, causing the matter to separate from the filter and flow distally in the first artery (e.g., the aorta) without entering the second artery (e.g., the carotid artery). This use of the moving parts may be practiced in combination with or separately from the breaking up of aggregated matter described herein.

Alternatively or additionally, control unit 32 moves moving parts 40 and 42 with respect to each other by driving the electrode to drive a current into the blood vessel that constricts the blood vessel. In some embodiments, the control unit constricts the blood vessel by driving a current having a frequency of between 40 Hz and 70 Hz. For some applications, the current has an amplitude of between 5 mA and 20 mA, e.g., between 8 mA and 15 mA. In some embodiments, a current having ten pulses to twenty pulses, e.g., thirteen pulses to seventeen pulses, per cardiac cycle, is driven into the blood vessel to constrict the blood vessel.

In all other aspects, filter 20 is generally as described with respect to FIG. 1.

Reference is now made to FIGS. 3A-B, which show filter 20 having two or more moving parts 40 and 42 implanted in blood vessel 22. FIG. 3A shows the filter disposed inside the blood vessel when the blood vessel is not dilated. In FIG. 3B, a current has been driven into the blood vessel that dilates the blood vessel by increasing nitric oxide (NO) secretion by the wall, causing moving part 40 to move in the direction of arrow 50 and moving part 42 to move in the direction of arrow 52.

For some applications, as shown, moving parts 40 and 42 are fundamentally separate elements, which are coupled together by a coupling mechanism (coupling mechanism not shown). For other applications, moving parts 40 and 42 are different portions of an integral whole, which flexes in response to the dilation and contraction of blood vessel 22. In either case, moving parts 40 and 42 are typically elastically coupled to each other, such that a rest position of moving parts 40 and 42 is generally as shown in FIG. 3B, and the elastic coupling (e.g., a spring or spring equivalent) is compressed in order to re-achieve the state shown in FIG. 3A. In this manner, electrodes 24 typically remain coupled to blood vessel 22 throughout all or a substantial portion of the cardiac cycle.

It is noted that control unit 32, as well as other apparatus described herein, may be powered by an integrated battery, or may receive energy wirelessly from another source, such as an RF or ultrasound transfer disposed outside of the subject's body.

In an embodiment, the filter described herein is integrated with a counterpulsation device, for example an intra-aortic counterpulsation device such as is described in U.S. patent application Ser. No. 12/023,896 to Gross et al., filed Jan. 31, 2008, entitled, “Intra-aortic electrical counterpulsation,” which is incorporated herein by reference. In an embodiment, the filter described herein is integrated with a counterpulsation device, for example an intra-aortic counterpulsation device such as is described in a PCT patent application to Gross et al., filed on even date herewith, entitled, “Intra-aortic electrical counterpulsation,” which is incorporated herein by reference. In some embodiments, the techniques described herein are practiced in combination with techniques described in PCT Publication WO 07/013065 to Gross, which is incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a filter, configured to be implanted in a blood vessel of a subject;
an electrode, configured to be disposed in a vicinity of the blood vessel; and
a control unit, configured to break up matter that aggregates on the filter, by driving the electrode to drive a current into the blood vessel and configuring the signal to increase nitric oxide (NO) secretion by the wall.

2. (canceled)

3. The apparatus according to claim 1, wherein the matter includes an embolus, and wherein the control unit is configured to break up the embolus by driving the electrode to apply the current to the wall of the blood vessel.

4. The apparatus according to claim 1, wherein the matter includes fibrotic material that adheres to the filter, and wherein the control unit is configured to break up the fibrotic material by driving the electrode to apply the current to the wall of the blood vessel.

5. (canceled)

6. The apparatus according to claim 1, wherein the electrode is configured to be disposed on the filter.

7-9. (canceled)

10. The apparatus according to claim 1, further comprising a detector configured to detect an aggregation of the matter on the filter, wherein the control unit is configured to drive the electrode to apply the current in response to the detector detecting the aggregation of the matter.

11-13. (canceled)

14. The apparatus according to claim 10, wherein the control unit is configured to drive the electrode to apply the current in accordance with a duty cycle, and wherein the control unit is configured to set the duty cycle such that the current is applied during fewer than 10 minutes of at least one hour of a day.

15. The apparatus according to claim 10, wherein the control unit is configured to drive the electrode to apply the current in accordance with a duty cycle, and wherein the control unit is configured to set the duty cycle such that the current is applied at least once during each of six hours of a day.

16. The apparatus according to claim 1, wherein the control unit is configured to break up the matter by driving the electrode to apply a current having an amplitude of between 1 mA and 5 mA.

17. (canceled)

18. The apparatus according to claim 1, wherein the control unit is configured to break up the matter by driving the electrode to apply a current having a frequency of between 5 Hz and 20 Hz.

19. (canceled)

20. The apparatus according to claim 1, wherein the control unit is configured to break up the matter by driving the electrode to apply a current having two pulses to eight pulses per cardiac cycle.

21. (canceled)

22. Apparatus, comprising:
a filter having two or more moving parts and configured to be implanted in a blood vessel of the subject;
an electrode, configured to be disposed in a vicinity of the blood vessel; and
a control unit, configured to move the moving parts with respect to each other by driving the electrode to drive a current into the blood vessel that changes a diameter of the blood vessel.

23. (canceled)

24. The apparatus according to claim 22, wherein the electrode is configured to be disposed on the filter.

25-29. (canceled)

30. The apparatus according to claim 22, wherein the control unit is configured to drive the electrode to drive a current into the blood vessel that dilates the blood vessel by increasing nitric oxide (NO) secretion by a wall of the blood vessel.

31. The apparatus according to claim 30, wherein the control unit is configured to dilate the blood vessel by driving the electrode to apply a current having an amplitude of between 1 mA and 5 mA.

32. (canceled)

33. The apparatus according to claim 30, wherein the control unit is configured to dilate the blood vessel by driving the electrode to apply a current having a frequency of between 5 Hz and 20 Hz.

34-36. (canceled)

37. The apparatus according to claim 22, wherein the control unit is configured to drive the electrode to drive a current into the blood vessel that constricts the blood vessel.

38. The apparatus according to claim 37, wherein the control unit is configured to constrict the blood vessel by driving the electrode to apply a current having a frequency of between 40 Hz and 70 Hz.

39. The apparatus according to claim 37, wherein the control unit is configured to constrict the blood vessel by driving the electrode to apply a current having an amplitude of between 5 mA and 20 mA.

40. (canceled)

41. The apparatus according to claim 37, wherein the control unit is configured to constrict the blood vessel by driving the electrode to apply a current having ten pulses to twenty pulses per cardiac cycle of the subject

42. (canceled)

43. The apparatus according to claim 22, wherein the control unit is configured to break up matter that aggregates on the filter by moving the moving parts with respect to each other.

44-88. (canceled)

89. Apparatus, comprising:
a filter having two or more moving parts and configured to be implanted in a blood vessel of the subject; and
a control unit, configured to clean the filter while the filter is in the subject, by moving the moving parts with respect to each other.
